(19) United States
(12) Patent Application Publication
(10) Pub. No.: US 2026/0224654 A1
(43) Pub. Date: Aug. 6, 2026

(54) METHODS AND SYSTEMS FOR DETERMINING PHYSIOLOGICAL STATES BY SALINITY OF BODILY FLUID SECRETIONS AND CRYSTALLIZATION IMAGING

(71) Applicant: Ovulio Corporation, Minnetonka, MN (US)

(72) Inventors: Serhii Zatsarynin, Minnetonka, MN (US); Yevginy Galyuk, Sherman Oaks, CA (US); Ihor Kovalenko, Minnetonka, MN (US)

(73) Assignee: Ovulio Corporation, Minnetonka, MN (US)

(21) Appl. No.: 19/530,617

(22) Filed: Feb. 5, 2026

Related U.S. Application Data
(60) Provisional application No. 63/754,020, filed on Feb. 5, 2025.

(51) Int. Cl. A61K 38/00; A61K 9/00; A61K 31/506

(52) U.S. Cl. CPC A61K 38/00; A61K 9/0019; A61K 31/506; A61K 2300/00

(57) ABSTRACT

A ferning pattern of a sample of a fluid collected from a subject are compared to a database of diagnostic ferning patterns indicative of adverse health condition to determine the health status of the subject. Cancer targets and salt complexes denoued for the first time drive the objects of the present inventions, which likewise are imaged by the instant hardware, owned by Ovulio, Inc. of Minnesota, USA.

### CROSS-REFERENCE TO RELATED APPLICATIONS

The present application claims priority to U.S. Provisional Application No. 63/754,020, filed Feb. 5, 2025, the contents of which are incorporated herein by reference in their entirety.

### BACKGROUND OF THE INVENTION

Saliva ferning, characterized by fern-like crystallization patterns observable in dried saliva viewed under a microscope, is a widely recognized phenomenon used for ovulation prediction. This pattern correlates strongly with hormonal changes, particularly fluctuations in estrogen levels during the menstrual cycle (Jaypee Digital, [5]). While saliva ferning is primarily driven by these hormonal variations, emerging evidence suggests that physiological systems regulating fluid balance, electrolyte composition, and salivary gland function also contribute significantly to its occurrence.

The Renin-Angiotensin-Aldosterone System (RAAS) plays a pivotal role in systemic and local electrolyte regulation, impacting the ionic balance of saliva. Through its key components, including aldosterone, the RAAS modulates sodium reabsorption and potassium excretion, thus influencing the biochemical environment essential for ferning (Cleveland Clinic, [2]). Additionally, the Kallikrein-Kinin System (KKS), via the vasoactive peptide bradykinin, enhances fluid secretion and promotes vascular permeability, contributing to the electrolyte dynamics in saliva (SpringerLink, [1]). These two systems operate in a counterbalancing manner to regulate vascular tone, fluid homeostasis, and glandular secretions, all of which are integral to the formation of saliva ferning patterns.

Angiotensin-Converting Enzyme (ACE) serves as a critical regulatory link between the RAAS and KKS. ACE converts angiotensin I to angiotensin II, a potent vasoconstrictor that activates the RAAS, while simultaneously degrading bradykinin, thus attenuating the effects of the KKS (Cleveland Clinic, [2]). Localized activity of ACE within salivary glands further modulates the interplay between these systems, indirectly influencing saliva production and electrolyte composition. ACE inhibitors, commonly used for hypertension, have been shown to alter the salivary electrolyte balance and may indirectly impact ferning patterns (Cleveland Clinic, [2]).

Hormonal influences, particularly estrogen and progesterone, further regulate these systemic mechanisms. Estrogen, which peaks during ovulation, enhances water and electrolyte retention, thereby increasing the concentration of sodium and chloride in saliva, key factors in the crystallization process (CEGH, [3]). Progesterone, which rises after ovulation, counteracts this effect by altering electrolyte secretion and fluid dynamics, leading to the reduction or absence of ferning patterns (Jaypee Digital, [5]). Estrogen also modulates RAAS activity by upregulating angiotensinogen production, further linking hormonal fluctuations to the systemic regulation of salivary properties (CEGH, [3]).

Locally, components of the RAAS and KKS expressed in salivary glands regulate blood flow, electrolyte transport, and glandular secretion. Bradykinin, as part of the KKS, increases vascular permeability and salivary gland output, further impacting the microstructural changes required for the crystallization of mucoproteins and salts in saliva (SpringerLink, [1]; Verywell Health, [4]). These processes are essential for the formation of the fern-like patterns observed during ovulation.

While the precise mechanisms remain incompletely understood, it is evident that saliva ferning arises from a complex interplay of hormonal, systemic, and local regulatory factors. The involvement of the RAAS, ACE, and KKS highlights the multifaceted nature of the phenomenon and underscores the potential for innovative diagnostic or therapeutic applications targeting these systems.

Further investigation into these relationships may lead to advancements in reproductive health diagnostics, salivary gland physiology, and electrolyte regulation technologies.

The regulation of fluid and electrolyte balance is critical to human health, with the Renin-Angiotensin-Aldosterone System (RAAS), Angiotensin-Converting Enzyme (ACE), and the Kallikrein-Kinin System (KKS) playing pivotal roles. Dysregulation of these systems can result in altered electrolyte excretion in mucosal and exocrine fluids, such as saliva or vaginal secretions. These disruptions are linked to various physiological and pathological conditions, each with distinct mechanisms and diagnostic implications.

Chronic stress or overactivation of the sympathetic nervous system can enhance RAAS activity, leading to systemic sodium retention while paradoxically increasing localized sodium chloride excretion in salivary secretions. This phenomenon results in hypertonic saliva, contributing to discomfort and oral dryness (Cleveland Clinic—Renin-Angiotensin-Aldosterone System (RAAS)).

In cystic fibrosis (CF), mutations in the CFTR gene impair chloride transport, causing compensatory increases in sodium secretion. This results in thickened mucus and elevated sodium chloride levels in bodily fluids, including saliva. Sweat chloride testing remains the gold standard for diagnosing CF, supported by genetic testing (Cleveland Clinic—Cystic Fibrosis).

Hyperaldosteronism, characterized by excessive aldosterone production, disrupts systemic and local electrolyte homeostasis. While it causes sodium retention and potassium excretion systemically, it may paradoxically increase sodium secretion in exocrine fluids. Diagnostic measures include aldosterone-to-renin ratio testing and adrenal imaging (Cleveland Clinic—Hyperaldosteronism).

In autoimmune diseases like Sjögren's syndrome, inflammatory damage to salivary glands reduces electrolyte reabsorption, resulting in hypertonic saliva and significant patient morbidity, including dental decay and difficulty swallowing (Johns Hopkins Sjögren's Center).

Diabetes Mellitus is another condition that indirectly impacts mucus salinity. Elevated blood glucose levels in diabetes lead to dehydration due to osmotic diuresis, a process in which excess glucose in the blood draws water into the urine. This dehydration concentrates electrolytes such as sodium and chloride in bodily fluids, including saliva and mucus. In diabetic ketoacidosis (DKA), a severe complication of diabetes, elevated levels of ketones and imbalances in chloride and sodium further exacerbate these effects. These changes in mucus salinity can contribute to the characteristic symptoms of dry mouth and thickened saliva seen in diabetes. Diagnostic tools for diabetes include fasting glucose tests, HbA1c measurements, and ketone testing for DKA (Medicinenet—How to Tell if You Are Dehydrated).

Chronic kidney disease (CKD) impairs renal function, leading to compensatory activation of RAAS. This results in altered electrolyte secretion pathways, such as increased sodium chloride levels in saliva. Diagnostic tools include GFR measurement and serum creatinine analysis (National Kidney Foundation).

Polycystic ovary syndrome (PCOS) involves hormonal imbalances that dysregulate RAAS activity, increasing sodium chloride levels in vaginal and salivary secretions during ovulatory phases. Diagnostic criteria include hormone testing and ultrasound imaging (Cleveland clinic—PCOS).

Pharmacological interventions like ACE inhibitors and angiotensin receptor blockers (ARBs) alter electrolyte dynamics in mucosal secretions. These medications reduce angiotensin II production and enhance bradykinin activity, increasing vascular permeability (TeachMePhysiology—The Renin-Angiotensin-Aldosterone System).

Infections or inflammatory conditions activating the KKS, such as bacterial vaginosis or sialadenitis, raise electrolyte content in secretions through bradykinin-mediated permeability changes (UpToDate—Bacterial Vaginosis).

Hormonal fluctuations, particularly estrogen and progesterone, directly and indirectly modulate electrolyte secretion. Estrogen enhances RAAS activity, promoting sodium chloride excretion during ovulatory phases (Jaypee Digital—Evaluation of Salivary Ferning for Predicting Ovulation).

Adrenal insufficiency, such as Addison's disease, reduces aldosterone production, impairing sodium retention and leading to compensatory excretion in saliva and other secretions (Cleveland Clinic—Addison's Disease).

Exocrine gland cancers disrupt normal glandular function, increasing salt content in secretions through duct compression, vascular changes, or inflammation. Diagnosis relies on imaging and biopsy (Johns Hopkins Medicine—Salivary Gland Cancer).

The interplay between RAAS, ACE, and KKS dysregulation and glandular function highlights the diagnostic potential of analyzing electrolyte-rich secretions like saliva and vaginal mucus. This invention aims to leverage these insights to develop advanced diagnostic tools and therapeutic interventions, addressing unmet needs in managing electrolyte imbalances and glandular dysfunction.

### SUMMARY OF THE INVENTION

The conditions discussed influence the salinity and electrolyte composition of saliva and bodily mucus through various mechanisms, impacting key electrolytes such as sodium (NaCl), chloride (Cl−), potassium (K+), phosphate (PO₄³−), and bicarbonate (HCO₃−). In cystic fibrosis (CF), mutations in the CFTR gene disrupt chloride and sodium transport, leading to thicker mucus with elevated salt content. Dehydration concentrates sodium, chloride, and potassium due to fluid loss, while hyperchloremia reflects elevated chloride levels, often alongside sodium, as a result of kidney dysfunction or metabolic acidosis. Diabetes mellitus induces osmotic diuresis, dehydrating the body and concentrating sodium and chloride in secretions; diabetic ketoacidosis further disrupts potassium levels. Chronic kidney disease (CKD) impairs electrolyte regulation, resulting in increased sodium, chloride, potassium, and phosphate in secretions as a compensatory response. Respiratory infections and chronic respiratory conditions, such as COPD and bronchiectasis, alter chloride, sodium, and bicarbonate levels in mucus through inflammation and epithelial disruption. In Sjögren's syndrome, autoimmune damage to salivary glands reduces fluid output, concentrating sodium, chloride, and potassium. Addison's disease, marked by hormonal insufficiency, impairs sodium retention and elevates potassium levels in secretions. Lastly, medications like diuretics and treatments inducing dehydration alter systemic electrolyte balance, reflecting in salivary and mucosal electrolyte composition. These variations in electrolyte levels across conditions provide valuable diagnostic insights into systemic and glandular dysfunctions.

The present invention teaches a novel and enhanced method and system for qualitative and quantitative analysis of mucous excreted electrolytes such as sodium (NaCl), chloride (Cl−), potassium (K+), phosphate (PO₄³−), and bicarbonate (HCO₃−) by optical imaging analysis of dried saliva samples using an artificial intelligence image recognition and processing algorithm capable of discerning otherwise imperceivable differences in crystal patterns found in dried saliva ferning crystallization and correlating said ferning crystallization patterns to know physical state or adverse health conditions previously diagnosed in human subjects.

Additionally, the present invention teaches a digital platform, consisting of hardware device plus digital mobile and web applications which are capable of collecting user health data metrics via user input and conversational artificial intelligence algorithm, referred to as second artificial intelligence algorithm and chronological user data. Furthermore, the present invention teaches a means for analyzing all subsequent sample images, user data and metrics, cross referencing historical data inputs with newly obtained data, identifying any anomalies or correlations in the images or user data and providing an AI generated conversational response alerting the user to any changes in their health state.

In addition to identifying specific health conditions by imaging and analyzing dried saliva and identifying crystallization and ferning phenomena which is caused by the body's natural processes, the present invention is capable of identifying specific crystallization patterns which are engineered or are caused by the addition of a saline solution reagent to a users saliva, and then imaged and analyzed by the device and artificial intelligence of the present invention.

Saliva contains a diverse range of proteins that serve various biological functions. Key proteins include amylase, which aids in the breakdown of starches; lysozyme, an enzyme with antibacterial properties; and lactoferrin, which binds iron and exhibits antimicrobial activity. Immunoglobulins (IgA, IgG, IgM) contribute to immune defense, while mucins (MUC5B, MUC7) lubricate and protect oral tissues. Histatins display antifungal and wound-healing properties, cystatins act as protease inhibitors to protect tissues, and proline-rich proteins (PRPs) play a role in mineral balance and protection against pathogens. The concentration of salt required to precipitate these proteins depends on their solubility and properties. For example, low salt concentrations (0.2ΓÇô0.5 M ammonium sulfate) can precipitate less soluble proteins like albumins, medium salt concentrations (0.5ΓÇô1.0 M ammonium sulfate) are effective for moderately soluble proteins such as globulins, and high salt concentrations (1.0ΓÇô2.0 M ammonium sulfate) are necessary for highly soluble proteins like mucins and immunoglobulins. Saliva also contains cancer-related proteins, including CA-125 (an ovarian cancer marker), cytokeratin's, p53 protein, and epidermal growth factor receptor (EGFR). The salt concentrations required for salting out these cancer-related proteins typically range from 0.5 to 1.5 M ammonium sulfate, though exact values vary based on experimental conditions and the specific properties of the proteins.

Additionally, it is understood and described in scientific literature that proteins of interest found in saliva of people with cancer include:
Cytokeratin 19 fragment (CYFRA 21-1): A soluble fragment of cytokeratin (CK) 19, which is overexpressed in saliva from people with oral squamous cell carcinoma (OSCC)
Tissue polypeptide antigen (TPS): A fragment of CK-18, which is a marker for rapid tumor growth
Cancer antigen 125 (CA-125 ): A tumor-associated mucin glycoprotein that is also seen in OSCC
CD44: A cell surface adhesion molecule that is highly expressed in advanced stages of OSCC
Cathepsin V: A protease that may be a useful biomarker for OSCC screening and diagnosis
Kallikrein 5: A protease that may be a useful biomarker for OSCC screening and diagnosis
ADAM9: A protease that may be a useful biomarker for OSCC screening and diagnosis
VEGF: A protein that may be useful for predicting breast cancer
EGF: A protein that may be useful for predicting breast cancer
CEA: A protein that may be useful for predicting breast cancer

Proteins in saliva can be used as biomarkers to help provide early diagnosis and monitor progression of cancer in patients.

The present invention teaches imaging and analyzing and identifying specific crystal patterns formed by combination of salts plus proteins found in dry saliva samples of mammals.

### DETAILED DESCRIPTION OF THE INVENTION

The present invention teaches systems and methods for establishing diagnostic protocols by means of imaged saliva ferning. The present invention consists of a digital electronic hardware device and a digital software platform comprising artificial intelligence models, cloud servers, mobile and web application, back end database, and an AI training software system.

The hardware system of the invention comprises a handheld device capable of receiving a sample of saliva onto an observation surface, allowing the sample to dry on an observation surface, a digital camera capable of taking a magnified image of the dried saliva sample on an observation surface, a lighting mechanism capable of illuminating the saliva sample on the observation surface, a micro processor capable of processing an image file, a Wi-Fi module capable of connecting to a wireless internet source, a bluetooth module capable of connecting to a bluetooth device, a microprocessor capable of transmitting data over the internet, a battery, a charger port and voltage regulating module, internal brackets to hold electronic components, an outer shell to protect electronic components.

The digital platform of the invention consists of an image recognition artificial intelligence algorithm capable of analyzing an image of patterns, discerning patterns found within the image, comparing imaged patterns against a database of known reference images of similar patterns, and assigning a match probability based on learned knowledge taught by a trained medical professional human who is knowledgeable in womens biological processes and saliva ferning patterns.

The digital platform of the present invention comprises a digital filter capable of diminishing imaging noise and amplifying patterns visible in images captured by the device camera of the present invention.

The digital platform of the invention consists of a second artificial intelligence algorithm capable of analyzing multiple data sets organized into a legible format recognizable and learnable by said AI algorithm. The purpose of the second artificial intelligence algorithm is to analyze specific user data metrics collected via the mobile or web application of the present invention, monitor current and historical data metrics collected via the mobile or web application of the present invention, find and identify correlations between current and historical data metrics collected via the mobile or web application of the present invention, find and identify any anomalies between current and historical data metrics collected via the mobile or web application of the present invention, find and identify any correlations between current data, historical data and newly received test data produced by the imaging artificial intelligence algorithm, find and identify any anomalies between current data, historical data and newly obtained test data produced by the imaging artificial intelligence, convert known correlations or anomalies found in the existing data into a language response, communicate a language response to the user via the mobile or web application in the form of a test result, communicate a language response in the form of a question to the user, process any language user inputs and cross reference against known pre taught IF scenarios, extrapolate know responses taught by IF scenarios and communicate said response to user via the mobile or web application, process, categorize and log any language user inputs for future reference, recall any user language inputs from memory, generate conversational responses based on knowledge recall and any user data inputs collected via mobile or web application, and user saliva sample images obtained by the camera of the device and processed by the imaging artificial intelligence of the present invention.

The present invention comprises multiple embodiments, which allow a greater flexibility in the overall architecture of the digital platform.

One embodiment of the present invention is a digital platform wherein the image processing artificial intelligence algorithm is hosted on a cloud server. In this embodiment, the device of the present invention captures an image of a dried saliva sample and transmits said image via a Wi-Fi internet connection to the server for further processing and analysis. After analysis, the image is transmitted to a database hosted on a separate server via an API key and stored for later retrieval by the second AI algorithm capable of analyzing the test result, cross referencing it with any relevant data in the user log and generating a conversational response to the user via the mobile or web application. In this embodiment of the present invention, both AI algorithms, and user data logs are stored on a cloud server and all backend processing is accomplished within the cloud. Once the second AI has processed the relevant data inputs, it generates a conversational result based on its findings and communicates it directly to the users mobile or web application in the form of a push notification.

In another embodiment of the present invention, the image processing algorithm is integrated directly into the application of the present invention and is hosted on the personal mobile device or computer of the end user. In this embodiment of the invention, the device of the present invention captures an image of the dried saliva sample, and transmits the image for further processing and analysis directly to the application of the present invention via WIFI or via Bluetooth. In this embodiment the second artificial intelligence algorithm is similarly integrated into the mobile or web application of the present invention and is stored directly on the device of the user.

The user health data collection method of the present invention comprises a mobile or web application with a hard coded onboarding questionnaire, question and answer forms, and a conversational artificial intelligence which is referred to as second artificial intelligence algorithm capable of conversational dialogue, IF logic comprehension and data analysis, a calendar capable of tracking user menstrual cycles and milestone dates, a notes form allowing users to note any changes in health state, diet, stress, physical activity, travel, medication use, drug use, alcohol use, medical intervention, adverse health event.

The analytical system and method of the present invention comprises an imaging camera capable of capturing a magnified high resolution image referred to as analyte image, a receptacle and imaging surface for the collection of a saliva sample, transmitting the raw image to a cloud server, processing analyte image through a digital image filter, allowing an image recognition and processing artificial intelligence algorithm to view the analyte image and its contents, allowing the image recognition and processing artificial intelligence algorithm to cross reference the analyte image contents against a database of known reference images which have been taught to the image recognition and processing algorithm previously, allow the image recognition and processing artificial intelligence algorithm to assign a similarity probability value to the analyte image based on known reference images, transmit the image into a users individual database, allow a second artificial intelligence algorithm to analyze the individual user database, allow the second artificial intelligence algorithm to find and establish any relevant correlations or anomalies in the individual users database, allow the second artificial intelligence algorithm to generate a conversational language response based on its analysis of the users database, allow the second artificial intelligence algorithm to send its generated conversational language response to the user via a mobile or web application.

Additionally, the analytical method of the present invention comprises a back end database of analyte images comprised of known health conditions which were verified by state of the art diagnostic tools and quantitative blood serum estrogen and progesterone levels organized in an understandable and legible format which is recognizable and processable by the second artificial intelligence algorithm, wherein, the second AI algorithm is capable of executing a comparative analysis and recall between the quantitative numerical values of blood serum samples and the correlating analyte images captured by the device of the present invention. After the second algorithm has analyzed and identified a correlation between blood serum data and analyte image data, the second ai algorithm generates a conversational language response in the form of a test result and transmits the result to the user via the mobile or web application of the present invention.

Furthermore, the analytical method of the present invention comprises a back end database consisting of user health and lifestyle questionnaire data, health and lifestyle data received via a notes form within the mobile or web application of the present invention, health and lifestyle data collected by the second ai algorithm through conversational chat dialogue with the user, chronological data collected via the cycle tracking calendar of the mobile or web application which has been organized into a understandable and legible format and processable by the second artificial intelligence algorithm and is accessible by the second ai algorithm. Wherein the second ai algorithm is capable of searching and identifying and recalling any correlation or anomalies between newly imputed data and historical data, generating a conversational language question or response based on its findings and transmitting the question or response to the user via the mobile or web application of the present invention.

The second artificial intelligence training method of the present invention comprises human clinical health metrics collected in the course of clinical trials and for the purpose of training the image recognition and processing artificial intelligence algorithm and the second artificial intelligence algorithm capable of analyzing a set of data and generating a conversational language response based on its findings.

One embodiment of the present invention requires the collection of a saliva sample from a human subject with a known health condition which affects RAAS, KKS and ACE such as cystic fibrosis, Sjögren's syndrome or chronic kidney disease, imaging the dried saliva sample by the device of the present invention and analyzing the dried saliva sample by the image recognition AI algorithm of the digital platform of the present invention over a period of time to establish a baseline control database.

In a different embodiment of the present invention, saliva samples are collected from patients with known cancer. In this embodiment of the invention, the saliva samples are treated with a saline solution reagent prior to the delivery of the sample into the device of the present invention, and further processing and analysis by the image processing artificial intelligence of the digital platform of the present invention.

The method of the present invention teaches the creation of an image database of dried saliva samples from patients with known conditions to create training reference images for the image processing artificial intelligence algorithm.

Another method of the present invention teaches creating an image database of dried saliva plus reagent samples from patients with known cancer conditions to create training reference images for the image processing artificial intelligence algorithm.

The known reference images are cataloged by known and confirmed medical conditions, and digitally inputted into the image processing artificial intelligence algorithm for pattern recognition. Each image is assigned a medical condition or health state by a medical professional overseeing the artificial intelligence algorithm training process.

Each image of a dried saliva sample or a dried saliva sample displays a unique crystal pattern caused by the combination of different proteins present in the saliva plus the different electrolytes present naturally in the saliva, or which have been added as a reagent to assist in the salting out of specific proteins of interest in patients with known cancer.

Known Salts for protein and enzyme precipitation are but not limited to:

Ammonium sulfate (NH₄)₂SO₄ is a widely utilized salt for “salting-out” proteins due to its exceptional solubility in aqueous solutions and minimal adverse effects on protein structure. By adjusting the degree of saturation, ammonium sulfate enables selective and gradual precipitation of proteins, preserving their functional integrity and making it a staple in biochemical and industrial processes.

Sodium chloride (NaCl) is commonly employed in simple salting-out methodologies and serves as a critical component for maintaining ionic strength within protein solutions, particularly in chromatography buffers, where it stabilizes proteins and enhances separation efficiency.

Potassium chloride (KCl) is frequently used in buffering systems to stabilize enzymes and proteins, effectively maintaining ionic strength without introducing divalent ions, which could interfere with protein activity.

Magnesium sulfate (MgSO4) is occasionally used for protein precipitation and serves as a stabilizer for certain enzymes, ensuring their structural and functional integrity. Calcium chloride (CaCl2) facilitates the targeted precipitation of specific proteins and the aggregation of nucleic acids for efficient removal, making it particularly valuable in DNA/protein isolation workflows.

Lithium chloride (LiCl) is a versatile salt used in RNA extraction and purification protocols, inhibiting the co-precipitation of proteins and carbohydrates and thereby enhancing RNA purity.

Sodium sulfate (Na₂SO₄) precipitates proteins at lower concentrations than sodium chloride, making it effective for less selective salting-out processes and bulk protein isolation.

Zinc sulfate (ZnSO4) is occasionally applied for stabilizing or precipitating specific proteins due to its selective interaction with protein molecules, providing precise control over protein separation.

### DETAILED DESCRIPTION OF THE INVENTION

As shown in the FIGS. 1 through 9 saliva crystallization and ferning is an occurring phenomenon in human male test subjects. Each pattern is indicative of a specific health condition which had been previously diagnosed.

The test subject's saliva was tested by state of the art methods such as ICP-MS and ELISA assay to establish the presence of protein enzymes as well as naturally present electrolytes and a data base was established. Similarly, saliva samples were collected for the purpose of imaging and analysis by the platform of the present invention. Results from ICP-MS and ELISA assay were compared and correlated to the images captured and processed by the platform of the present invention.

The correlated data was then assigned with the ferning and crystallization patterns imaged and processed by the device and digital platform of the present invention.

The manually assigned data and images were then used as training inputs for the image processing artificial intelligence, wherein the image processing artificial intelligence algorithm became capable of discerning each specific crystal pattern and assigning that pattern to a known condition which corresponded to the manually imputed health condition data, protein and electrolyte concentration data received from the ICP-MS and ELISA assay and finally, the saline reagent solutions used to salt out proteins from saliva samples of test subjects.

The method of the present invention teaches a plurality of salts in combination with specific proteins. A saline solution may comprise of 1 salt, 2 salts, 3 salts, 4 salts, 5 salts, 6 salts, 7 salts, 8 salts depending on the health condition and protein of interests which is being precipitated from saliva for imaging by the device of the present invention wherein the captured image is further processed by the image processing artificial intelligence algorithm of the present invention.

Saline solution reagents have been tested in multiple plurality combinations:

### Single Salt Combinations

1. Ammonium Sulfate (NH4)2SO4(NH_4)_2SO_4(NH4ΓÇï)2ΓÇïSO4ΓÇï
2. Sodium Chloride (NaCl)
3. Potassium Chloride (KCl)
4. Magnesium Sulfate (MgSO4)(MgSO_4)(MgSO4ΓÇï)
5. Calcium Chloride (CaCl2)(CaCl_2)(CaCl2ΓÇï)
6. Lithium Chloride (LiCl)
7. Sodium Sulfate (Na2SO4)(Na_2SO_4)(Na2ΓÇïSO4ΓÇï)
8. Zinc Sulfate (ZnSO4)(ZnSO_4)(ZnSO4ΓÇï)

Whereas each individual salt is mixed with pharmaceutical grade solvent to create a saline solution reagent and added to a saliva sample containing one of the following proteins of interest which had previously been identified in the saliva sample by ICP-MS or ELISA assay.
Cytokeratin 19 fragment (CYFRA 21-1)
Tissue polypeptide antigen (TPS)
Cancer antigen 125 (CA-125 )
CD44
Cathepsin V
Kallikrein 5
ADAM9
VEGF
EGF
CEA

The combination of saline solution plus saliva sample forces a unique crystal pattern which can be imaged, processed, identified and assigned a predetermined health condition which has been taught to the image processing artificial intelligence of the present invention.

### Two-Salt Combinations

9. Ammonium Sulfate (NH4)2SO4(NH_4)_2SO_4(NH4ΓÇï)2ΓÇïSO4ΓÇï, Sodium Chloride (NaCl)
10. Ammonium Sulfate (NH4)2SO4(NH_4)_2SO_4(NH4ΓÇï)2ΓÇïSO4ΓÇï, Potassium Chloride (KCl)
11. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï)
12. Ammonium Sulfate (NH4)2SO4(NH_4)_2SO_4(NH4ΓÇï)2ΓÇïSO4ΓÇï, Calcium Chloride (CaCl2)(CaCl_2)(CaCl2ΓÇï)
13. Ammonium Sulfate (NH4)2SO4(NH_4)_2SO_4(NH4ΓÇï)2ΓÇïSO4ΓÇï, Lithium Chloride (LiCl)
14. Ammonium Sulfate (NH4)2SO4(NH_4)_2SO_4(NH4ΓÇï)2ΓÇïSO4ΓÇï, Sodium Sulfate (Na2SO4)(Na_2SO_4)(Na2ΓÇïSO4ΓÇï)
15. Ammonium Sulfate (NH4)2SO4(NH_4)_2SO_4(NH4ΓÇï)2ΓÇïSO4ΓÇï, Zinc Sulfate (ZnSO4)(ZnSO_4)(ZnSO4ΓÇï)
16. Sodium Chloride (NaCl), Potassium Chloride (KCl)
17. Sodium Chloride (NaCl), Magnesium Sulfate (MgSO4)(MgSO_4)(MgSO4ΓÇï)
18. Sodium Chloride (NaCl), Calcium Chloride (CaCl2)(CaCl_2)(CaCl2ΓÇï)
19. Sodium Chloride (NaCl), Lithium Chloride (LiCl)
20. Sodium Chloride (NaCl), Sodium Sulfate (Na2SO4)(Na_2SO_4)(Na2ΓÇïSO4ΓÇï)
21. Sodium Chloride (NaCl), Zinc Sulfate (ZnSO4)(ZnSO_4)(ZnSO4ΓÇï)
22. Potassium Chloride (KCl), Magnesium Sulfate (MgSO4)(MgSO_4)(MgSO4ΓÇï)
23. Potassium Chloride (KCl), Calcium Chloride (CaCl2)(CaCl_2)(CaCl2ΓÇï)
24. Potassium Chloride (KCl), Lithium Chloride (LiCl)
25. Potassium Chloride (KCl), Sodium Sulfate (Na2SO4)(Na_2SO_4)(Na2ΓÇïSO4ΓÇï)
26. Potassium Chloride (KCl), Zinc Sulfate (ZnSO4)(ZnSO_4)(ZnSO4ΓÇï)
27. Magnesium Sulfate (MgSO4)(MgSO_4)(MgSO4ΓÇï), Calcium Chloride (CaCl2)(CaCl_2) (CaCl2ΓÇï)
28. Magnesium Sulfate (MgSO4)(MgSO_4)(MgSO4ΓÇï), Lithium Chloride (LiCl)
29. Magnesium Sulfate (MgSO4)(MgSO_4)(MgSO4ΓÇï), Sodium Sulfate (Na2SO4) (Na_2SO_4)(Na2ΓÇïSO4ΓÇï)
30. Magnesium Sulfate (MgSO4)(MgSO_4)(MgSO4ΓÇï), Zinc Sulfate (ZnSO4)(ZnSO_4) (ZnSO4ΓÇï)
31. Calcium Chloride (CaCl2)(CaCl_2)(CaCl2ΓÇï), Lithium Chloride (LiCl)
32. Calcium Chloride (CaCl2)(CaCl_2)(CaCl2ΓÇï), Sodium Sulfate (Na2SO4)(Na_2SO_4) (Na2ΓÇïSO4ΓÇï)
33. Calcium Chloride (CaCl2)(CaCl_2)(CaCl2ΓÇï), Zinc Sulfate (ZnSO4)(ZnSO_4)(ZnSO4ΓÇï)
34. Lithium Chloride (LiCl), Sodium Sulfate (Na2SO4)(Na_2SO_4)(Na2ΓÇïSO4ΓÇï)
35. Lithium Chloride (LiCl), Zinc Sulfate (ZnSO4)(ZnSO_4)(ZnSO4ΓÇï)
36. Sodium Sulfate (Na2SO4)(Na_2SO_4)(Na2ΓÇïSO4ΓÇï), Zinc Sulfate (ZnSO4)(ZnSO_4) (ZnSO4ΓÇï)

Whereas two salts are mixed with pharmaceutical grade solvent to create a saline solution reagent and added to a saliva sample containing one of the following proteins of interest which had previously been identified in the saliva sample by ICP-MS or ELISA assay.
Cytokeratin 19 fragment (CYFRA 21-1)
Tissue polypeptide antigen (TPS)
Cancer antigen 125 (CA-125 )
CD44
Cathepsin V
Kallikrein 5
ADAM9
VEGF
EGF
CEA

The combination of saline solution plus a saliva sample forces a unique crystal pattern which can be imaged, processed, identified and assigned a predetermined health condition which has been taught to the image processing artificial intelligence of the present invention.

### Three-Salt Combinations

37. Ammonium Sulfate (NH4)2SO4(NH_4)_2SO_4(NH4ΓÇï)2ΓÇïSO4ΓÇï, Sodium Chloride (NaCl), Potassium Chloride (KCl)
38. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï)
39. Ammonium Sulfate (NH4)2SO4(NH_4)_2SO_4(NH4ΓÇï)2ΓÇïSO4ΓÇï, Sodium Chloride (NaCl), Calcium Chloride (CaCl2)(CaCl_2)(CaCl2ΓÇï)
40. Ammonium Sulfate (NH4)2SO4(NH_4)_2SO_4(NH4ΓÇï)2ΓÇïSO4ΓÇï, Sodium Chloride (NaCl), Lithium Chloride (LiCl)
41. Ammonium Sulfate (NH4)2SO4(NH_4)_2SO_4(NH4γÇï)2γÇïSO4γÇï, Sodium Chloride (NaCl), Sodium Sulfate (Na2SO4)(Na_2SO_4)(Na2ΓÇïSO4ΓÇï)
42. Ammonium Sulfate (NH4)2SO4(NH_4)_2SO_4(NH4ΓÇï)2ΓÇïSO4ΓÇï, Sodium Chloride (NaCl), Zinc Sulfate (ZnSO4)(ZnSO_4)(ZnSO4ΓÇï)
43. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï)
44. Ammonium Sulfate (NH4)2SO4(NH_4)_2SO_4(NH4ΓÇï)2ΓÇïSO4ΓÇï, Potassium Chloride (KCl), Calcium Chloride (CaCl2)(CaCl_2)(CaCl2ΓÇï)
45. Ammonium Sulfate (NH4)2SO4(NH_4)_2SO_4(NH4ΓÇï)2ΓÇïSO4ΓÇï, Potassium Chloride (KCl), Lithium Chloride (LiCl)
46. Ammonium Sulfate (NH4)2SO4(NH_4)_2SO_4(NH4ΓÇï)2ΓÇïSO4ΓÇï, Potassium Chloride (KCl), Sodium Sulfate (Na2SO4)(Na_2SO_4)(Na2ΓÇïSO4ΓÇï)
47. Ammonium Sulfate (NH4)2SO4(NH_4)_2SO_4(NH4ΓÇï)2ΓÇïSO4ΓÇï, Potassium Chloride (KCl), Zinc Sulfate (ZnSO4)(ZnSO_4)(ZnSO4ΓÇï)
48. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Calcium Chloride (CaCl2)(CaCl_2)(CaCl2ΓÇï)
49. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Lithium Chloride (LiCl)
50. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Sodium Sulfate (Na2SO4)(Na_2SO_4)(Na2ΓÇïSO4ΓÇï) 51. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Zinc Sulfate (ZnSO4)(ZnSO_4)(ZnSO4ΓÇï)
52. Ammonium Sulfate (NH4)2SO4(NH_4)_2SO_4(NH4ΓÇï)2ΓÇïSO4ΓÇï, Calcium Chloride (CaCl2)(CaCl_2)(CaCl2ΓÇï), Lithium Chloride (LiCl)
53. Ammonium Sulfate (NH4)2SO4(NH_4)_2SO_4(NH4ΓÇï)2ΓÇïSO4ΓÇï, Calcium Chloride (CaCl2)(CaCl_2)(CaCl2ΓÇï), Sodium Sulfate (Na2SO4)(Na_2SO_4)(Na2ΓÇïSO4ΓÇï)
54. Ammonium Sulfate (NH4)2SO4(NH_4)_2SO_4(NH4ΓÇï)2ΓÇïSO4ΓÇï, Calcium Chloride (CaCl2)(CaCl_2)(CaCl2ΓÇï), Zinc Sulfate (ZnSO4)(ZnSO_4)(ZnSO4ΓÇï)
55. Ammonium Sulfate (NH4)2SO4(NH_4)_2SO_4(NH4ΓÇï)2ΓÇïSO4ΓÇï, Lithium Chloride (LiCl), Sodium Sulfate (Na2SO4)(Na_2SO_4)(Na2ΓÇïSO4ΓÇï)
56. Ammonium Sulfate (NH4)2SO4(NH_4)_2SO_4(NH4ΓÇï)2ΓÇïSO4ΓÇï, Lithium Chloride (LiCl), Zinc Sulfate (ZnSO4)(ZnSO_4)(ZnSO4ΓÇï)
57. Ammonium Sulfate (NH4)2SO4(NH_4)_2SO_4(NH4ΓÇï)2ΓÇïSO4ΓÇï, Sodium Sulfate (Na2SO4)(Na_2SO_4)(Na2ΓÇïSO4ΓÇï), Zinc Sulfate (ZnSO4)(ZnSO_4)(ZnSO4ΓÇï)
58. Sodium Chloride (NaCl), Potassium Chloride (KCl), Magnesium Sulfate (MgSO4) (MgSO_4)(MgSO4ΓÇï)
59. Sodium Chloride (NaCl), Potassium Chloride (KCl), Calcium Chloride (CaCl2)(CaCl_2) (CaCl2ΓÇï)
60. Sodium Chloride (NaCl), Potassium Chloride (KCl), Lithium Chloride (LiCl)
61. Sodium Chloride (NaCl), Potassium Chloride (KCl), Sodium Sulfate (Na2SO4)(Na_2SO_4) (Na2ΓÇïSO4ΓÇï)
62. Sodium Chloride (NaCl), Potassium Chloride (KCl), Zinc Sulfate (ZnSO4)(ZnSO_4) (ZnSO4ΓÇï)
63. Sodium Chloride (NaCl), Magnesium Sulfate (MgSO4)(MgSO_4)(MgSO4ΓÇï), Calcium Chloride (CaCl2)(CaCl_2)(CaCl2ΓÇï)
64. Sodium Chloride (NaCl), Magnesium Sulfate (MgSO4)(MgSO_4)(MgSO4ΓÇï), Lithium Chloride (LiCl)
65. Sodium Chloride (NaCl), Magnesium Sulfate (MgSO4)(MgSO_4)(MgSO4ΓÇï), Sodium Sulfate (Na2SO4)(Na_2SO_4)(Na2ΓÇïSO4ΓÇï)
66. Sodium Chloride (NaCl), Magnesium Sulfate (MgSO4)(MgSO_4)(MgSO4ΓÇï), Zinc Sulfate (ZnSO4)(ZnSO_4)(ZnSO4ΓÇï)
67. Sodium Chloride (NaCl), Calcium Chloride (CaCl2)(CaCl_2)(CaCl2ΓÇï), Lithium Chloride (LiCl)
68. Sodium Chloride (NaCl), Calcium Chloride (CaCl2)(CaCl_2)(CaCl2ΓÇï), Sodium Sulfate (Na2SO4)(Na_2SO_4)(Na2ΓÇïSO4ΓÇï)
69. Sodium Chloride (NaCl), Calcium Chloride (CaCl2)(CaCl_2)(CaCl2ΓÇï), Zinc Sulfate (ZnSO4)(ZnSO_4)(ZnSO4ΓÇï)
70. Sodium Chloride (NaCl), Lithium Chloride (LiCl), Sodium Sulfate (Na2SO4)(Na_2SO_4) (Na2ΓÇïSO4ΓÇï)
71. Sodium Chloride (NaCl), Lithium Chloride (LiCl), Zinc Sulfate (ZnSO4)(ZnSO_4) (ZnSO4ΓÇï)
72. Sodium Chloride (NaCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)
73. Potassium Chloride (KCl), Magnesium Sulfate (MgSO4)(MgSO_4)(MgSO4ΓÇï), Calcium Chloride (CaCl2)(CaCl_2)(CaCl2ΓÇï)
74. Potassium Chloride (KCl), Magnesium Sulfate (MgSO4)(MgSO_4)(MgSO4ΓÇï), Lithium Chloride (LiCl)
75. Potassium Chloride (KCl), Magnesium Sulfate (MgSO4)(MgSO_4)(MgSO4ΓÇï), Sodium Sulfate (Na2SO4)(Na_2SO_4)(Na2ΓÇïSO4ΓÇï)
76. Potassium Chloride (KCl), Magnesium Sulfate (MgSO4)(MgSO_4)(MgSO4ΓÇï), Zinc Sulfate (ZnSO4)(ZnSO_4)(ZnSO4ΓÇï)
77. Potassium Chloride (KCl), Calcium Chloride (CaCl2)(CaCl_2)(CaCl2ΓÇï), Lithium Chloride (LiCl)
78. Potassium Chloride (KCl), Calcium Chloride (CaCl2)(CaCl_2)(CaCl2ΓÇï), Sodium Sulfate (Na2SO4)(Na_2SO_4)(Na2ΓÇïSO4ΓÇï)
79. Potassium Chloride (KCl), Calcium Chloride (CaCl2)(CaCl_2)(CaCl2ΓÇï), Zinc Sulfate (ZnSO4)(ZnSO_4)(ZnSO4ΓÇï)
80. Potassium Chloride (KCl), Lithium Chloride (LiCl), Sodium Sulfate (Na2SO4)(Na_2SO_4) (Na2ΓÇïSO4ΓÇï)
81. Potassium Chloride (KCl), Lithium Chloride (LiCl), Zinc Sulfate (ZnSO4)(ZnSO_4) (ZnSO4ΓÇï)
82. Potassium Chloride (KCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)
83. Magnesium Sulfate (MgSO4)(MgSO_4)(MgSO4ΓÇï), Calcium Chloride (CaCl2)(CaCl_2) (CaCl2ΓÇï), Lithium Chloride (LiCl)
84. Magnesium Sulfate (MgSO4)(MgSO_4)(MgSO4ΓÇï), Calcium Chloride (CaCl2)(CaCl_2) (CaCl2ΓÇï), Sodium Sulfate (Na2SO4)(Na_2SO_4)(Na2ΓÇïSO4ΓÇï)
85. Magnesium Sulfate (MgSO4)(MgSO_4)(MgSO4ΓÇï), Calcium Chloride (CaCl2)(CaCl_2) (CaCl2ΓÇï), Zinc Sulfate (ZnSO4)(ZnSO_4)(ZnSO4ΓÇï)
86. Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï)
87. Magnesium Sulfate (MgSO4)(MgSO_4)(MgSO4ΓÇï), Lithium Chloride (LiCl), Zinc Sulfate (ZnSO4)(ZnSO_4)(ZnSO4ΓÇï)
88. Magnesium Sulfate (MgSO4)(MgSO_4)(MgSO4ΓÇï), Sodium Sulfate (Na2SO4) (Na_2SO_4)(Na2ΓÇïSO4ΓÇï), Zinc Sulfate (ZnSO4)(ZnSO_4)(ZnSO4ΓÇï)
89. Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï)
90. Calcium Chloride (CaCl2)(CaCl_2)(CaCl2ΓÇï), Lithium Chloride (LiCl), Zinc Sulfate (ZnSO4)(ZnSO_4)(ZnSO4ΓÇï)
91. Calcium Chloride (CaCl2)(CaCl_2)(CaCl2ΓÇï), Sodium Sulfate (Na2SO4)(Na_2SO_4) (Na2ΓÇïSO4ΓÇï), Zinc Sulfate (ZnSO4)(ZnSO_4)(ZnSO4ΓÇï)
92. Lithium Chloride (LiCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)

Whereas 3 salts are combined and mixed with pharmaceutical grade solvent to create a saline solution reagent and added to a saliva sample containing one of the following proteins of interest which had previously been identified in the saliva sample by ICP-MS or ELISA assay.
Cytokeratin 19 fragment (CYFRA 21-1)
Tissue polypeptide antigen (TPS)
Cancer antigen 125 (CA-125 )
CD44
Cathepsin V
Kallikrein 5
ADAM9
VEGF
EGF
CEA

The combination of saline solution plus saliva sample forces a unique crystal pattern which can be imaged, processed, identified and assigned a predetermined health condition which has been taught to the image processing artificial intelligence of the present invention.

### Four-Salt Combinations

93. Ammonium Sulfate (NH4)2SO4(NH_4)_2SO_4(NH4γÇï)2γÇïSO4γÇï, Sodium Chloride (NaCl), Potassium Chloride (KCl), Magnesium Sulfate (MgSO4)(MgSO_4)(MgSO4ΓÇï)
94. Ammonium Sulfate (NH4)2SO4(NH_4)_2SO_4(NH4ΓÇï)2ΓÇïSO4ΓÇï, Sodium Chloride (NaCl), Potassium Chloride (KCl), Calcium Chloride (CaCl2)(CaCl_2)(CaCl2ΓÇï)
95. Ammonium Sulfate (NH4)2SO4(NH_4)_2SO_4(NH4ΓÇï)2ΓÇïSO4ΓÇï, Sodium Chloride (NaCl), Potassium Chloride (KCl), Lithium Chloride (LiCl)
96. Ammonium Sulfate (NH4)2SO4(NH_4)_2SO_4(NH4ΓÇï)2ΓÇïSO4ΓÇï, Sodium Chloride (NaCl), Potassium Chloride (KCl), Sodium Sulfate (Na2SO4)(Na_2SO_4)(Na2ΓÇïSO4ΓÇï)
97. Ammonium Sulfate (NH4)2SO4(NH_4)_2SO_4(NH4ΓÇï)2ΓÇïSO4ΓÇï, Sodium Chloride (NaCl), Potassium Chloride (KCl), Zinc Sulfate (ZnSO4)(ZnSO_4)(ZnSO4ΓÇï)
98. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Calcium Chloride (CaCl2) (CaCl_2)(CaCl2ΓÇï)
99. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Lithium Chloride (LiCl)
100. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Sodium Sulfate (Na2SO4) (Na_2SO_4)(Na2ΓÇïSO4ΓÇï)
101. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Zinc Sulfate (ZnSO4)(ZnSO_4) (ZnSO4ΓÇï)
102. Ammonium Sulfate (NH4)2SO4(NH_4)_2SO_4(NH4ΓÇï)2ΓÇïSO4ΓÇï, Sodium Chloride (NaCl), Calcium Chloride (CaCl2)(CaCl_2)(CaCl2ΓÇï), Lithium Chloride (LiCl)
103. Ammonium Sulfate (NH4)2SO4(NH_4)_2SO_4(NH4ΓÇï)2ΓÇïSO4ΓÇï, Sodium Chloride (NaCl), Calcium Chloride (CaCl2)(CaCl_2)(CaCl2ΓÇï), Sodium Sulfate (Na2SO4)(Na_2SO_4) (Na2ΓÇïSO4ΓÇï)
104. Ammonium Sulfate (NH4)2SO4(NH_4)_2SO_4(NH4ΓÇï)2ΓÇïSO4ΓÇï, Sodium Chloride (NaCl), Calcium Chloride (CaCl2)(CaCl_2)(CaCl2ΓÇï), Zinc Sulfate (ZnSO4)(ZnSO_4) (ZnSO4ΓÇï)
105. Ammonium Sulfate (NH4)2SO4(NH_4)_2SO_4(NH4ΓÇï)2ΓÇïSO4ΓÇï, Sodium Chloride (NaCl), Lithium Chloride (LiCl), Sodium Sulfate (Na2SO4)(Na_2SO_4)(Na2ΓÇïSO4ΓÇï)
106. Ammonium Sulfate (NH4)2SO4(NH_4)_2SO_4(NH4ΓÇï)2ΓÇïSO4ΓÇï, Sodium Chloride (NaCl), Lithium Chloride (LiCl), Zinc Sulfate (ZnSO4)(ZnSO_4)(ZnSO4ΓÇï)
107. Ammonium Sulfate (NH4)2SO4(NH_4)_2SO_4(NH4ΓÇï)2ΓÇïSO4ΓÇï, Sodium Chloride (NaCl), Sodium Sulfate (Na2SO4)(Na_2SO_4)(Na2ΓÇïSO4ΓÇï), Zinc Sulfate (ZnSO4) (ZnSO_4)(ZnSO4ΓÇï)
108. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Calcium Chloride (CaCl2)(CaCl_2)(CaCl2ΓÇï)
109. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Lithium Chloride (LiCl)
110. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Sodium Sulfate (Na2SO4)(Na_2SO_4)(Na2ΓÇïSO4ΓÇï)
111. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Zinc Sulfate (ZnSO4) (ZnSO_4)(ZnSO4ΓÇï)
112. Ammonium Sulfate (NH4)2SO4(NH_4)_2SO_4(NH4ΓÇï)2ΓÇïSO4ΓÇï, Potassium Chloride (KCl), Calcium Chloride (CaCl2)(CaCl_2)(CaCl2ΓÇï), Lithium Chloride (LiCl)
113. Ammonium Sulfate (NH4)2SO4(NH_4)_2SO_4(NH4ΓÇï)2ΓÇïSO4ΓÇï, Potassium Chloride (KCl), Calcium Chloride (CaCl2)(CaCl_2)(CaCl2ΓÇï), Sodium Sulfate (Na2SO4) (Na_2SO_4)(Na2ΓÇïSO4ΓÇï)
114. Ammonium Sulfate (NH4)2SO4(NH_4)_2SO_4(NH4ΓÇï)2ΓÇïSO4ΓÇï, Potassium Chloride (KCl), Calcium Chloride (CaCl2)(CaCl_2)(CaCl2ΓÇï), Zinc Sulfate (ZnSO4)(ZnSO_4) (ZnSO4ΓÇï)
115. Ammonium Sulfate (NH4)2SO4(NH_4)_2SO_4(NH4ΓÇï)2ΓÇïSO4ΓÇï, Potassium Chloride (KCl), Lithium Chloride (LiCl), Sodium Sulfate (Na2SO4)(Na_2SO_4) (Na2ΓÇïSO4ΓÇï)
116. Ammonium Sulfate (NH4)2SO4(NH_4)_2SO_4(NH4ΓÇï)2ΓÇïSO4ΓÇï, Potassium Chloride (KCl), Lithium Chloride (LiCl), Zinc Sulfate (ZnSO4)(ZnSO_4)(ZnSO4ΓÇï)
117. Ammonium Sulfate (NH4)2SO4(NH_4)_2SO_4(NH4ΓÇï)2ΓÇïSO4ΓÇï, Potassium Chloride (KCl), Sodium Sulfate (Na2SO4)(Na_2SO_4)(Na2ΓÇïSO4ΓÇï), Zinc Sulfate (ZnSO4) (ZnSO_4)(ZnSO4ΓÇï)
118. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Calcium Chloride (CaCl2)(CaCl_2)(CaCl2ΓÇï), Lithium Chloride (LiCl)
119. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Calcium Chloride (CaCl2)(CaCl_2)(CaCl2ΓÇï), Sodium Sulfate (Na2SO4)(Na_2SO_4)(Na2ΓÇïSO4ΓÇï)
120. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Calcium Chloride (CaCl2)(CaCl_2)(CaCl2ΓÇï), Zinc Sulfate (ZnSO4)(ZnSO_4)(ZnSO4ΓÇï)
121. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Lithium Chloride (LiCl), Sodium Sulfate (Na2SO4) (Na_2SO_4)(Na2ΓÇïSO4ΓÇï)
122. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Lithium Chloride (LiCl), Zinc Sulfate (ZnSO4) (ZnSO_4)(ZnSO4ΓÇï)
123. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Sodium Sulfate (Na2SO4)(Na_2SO_4) (Na2ΓÇïSO4ΓÇï), Zinc Sulfate (ZnSO4)(ZnSO_4)(ZnSO4ΓÇï)
124. Ammonium Sulfate (NH4)2SO4(NH_4)_2SO_4(NH4ΓÇï)2ΓÇïSO4ΓÇï, Calcium Chloride (CaCl2)(CaCl_2)(CaCl2ΓÇï), Lithium Chloride (LiCl), Sodium Sulfate (Na2SO4)(Na_2SO_4) (Na2ΓÇïSO4ΓÇï)
125. Ammonium Sulfate (NH4)2SO4(NH_4)_2SO_4(NH4ΓÇï)2ΓÇïSO4ΓÇï, Calcium Chloride (CaCl2)(CaCl_2)(CaCl2ΓÇï), Lithium Chloride (LiCl), Zinc Sulfate (ZnSO4)(ZnSO_4) (ZnSO4ΓÇï)
126. Ammonium Sulfate (NH4)2SO4(NH_4)_2SO_4(NH4ΓÇï)2ΓÇïSO4ΓÇï, Calcium Chloride (CaCl2)(CaCl_2)(CaCl2ΓÇï), Sodium Sulfate (Na2SO4)(Na_2SO_4)(Na2ΓÇïSO4ΓÇï), Zinc Sulfate (ZnSO4)(ZnSO_4)(ZnSO4ΓÇï)
127. Ammonium Sulfate (NH4)2SO4(NH_4)_2SO_4(NH4ΓÇï)2ΓÇïSO4ΓÇï, Lithium Chloride (LiCl), Sodium Sulfate (Na2SO4)(Na_2SO_4)(Na2ΓÇïSO4ΓÇï), Zinc Sulfate (ZnSO4) (ZnSO_4)(ZnSO4ΓÇï)
128. Sodium Chloride (NaCl), Potassium Chloride (KCl), Magnesium Sulfate (MgSO4) (MgSO_4)(MgSO4ΓÇï), Calcium Chloride (CaCl2)(CaCl_2)(CaCl2ΓÇï)
129. Sodium Chloride (NaCl), Potassium Chloride (KCl), Magnesium Sulfate (MgSO4) (MgSO_4)(MgSO4ΓÇï), Lithium Chloride (LiCl)
130. Sodium Chloride (NaCl), Potassium Chloride (KCl), Magnesium Sulfate (MgSO4) (MgSO_4)(MgSO4ΓÇï), Sodium Sulfate (Na2SO4)(Na_2SO_4)(Na2ΓÇïSO4ΓÇï)
131. Sodium Chloride (NaCl), Potassium Chloride (KCl), Magnesium Sulfate (MgSO4) (MgSO_4)(MgSO4ΓÇï), Zinc Sulfate (ZnSO4)(ZnSO_4)(ZnSO4ΓÇï)
132. Sodium Chloride (NaCl), Potassium Chloride (KCl), Calcium Chloride (CaCl2)(CaCl_2) (CaCl2ΓÇï), Lithium Chloride (LiCl)
133. Sodium Chloride (NaCl), Potassium Chloride (KCl), Calcium Chloride (CaCl2)(CaCl_2) (CaCl2ΓÇï), Sodium Sulfate (Na2SO4)(Na_2SO_4)(Na2ΓÇïSO4ΓÇï)
134. Sodium Chloride (NaCl), Potassium Chloride (KCl), Calcium Chloride (CaCl2)(CaCl_2) (CaCl2ΓÇï), Zinc Sulfate (ZnSO4)(ZnSO_4)(ZnSO4ΓÇï)
135. Sodium Chloride (NaCl), Potassium Chloride (KCl), Lithium Chloride (LiCl), Sodium Sulfate (Na2SO4)(Na_2SO_4)(Na2ΓÇïSO4ΓÇï)
136. Sodium Chloride (NaCl), Potassium Chloride (KCl), Lithium Chloride (LiCl), Zinc Sulfate (ZnSO4)(ZnSO_4)(ZnSO4ΓÇï)
137. Sodium Chloride (NaCl), Potassium Chloride (KCl), Sodium Sulfate (Na2SO4)(Na_2SO_4) (Na2ΓÇïSO4ΓÇï), Zinc Sulfate (ZnSO4)(ZnSO_4)(ZnSO4ΓÇï)
138. Sodium Chloride (NaCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl)
139. Sodium Chloride (NaCl), Magnesium Sulfate (MgSO4)(MgSO_4)(MgSO4ΓÇï), Calcium Chloride (CaCl2)(CaCl_2)(CaCl2ΓÇï), Sodium Sulfate (Na2SO4)(Na_2SO_4)(Na2ΓÇïSO4ΓÇï)
140. Sodium Chloride (NaCl), Magnesium Sulfate (MgSO4)(MgSO_4)(MgSO4ΓÇï), Calcium Chloride (CaCl2)(CaCl_2)(CaCl2ΓÇï), Zinc Sulfate (ZnSO4)(ZnSO_4)(ZnSO4ΓÇï)
141. Sodium Chloride (NaCl), Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï)
142. Sodium Chloride (NaCl), Magnesium Sulfate (MgSO4)(MgSO_4)(MgSO4ΓÇï), Lithium Chloride (LiCl), Zinc Sulfate (ZnSO4)(ZnSO_4)(ZnSO4ΓÇï)
143. Sodium Chloride (NaCl), Magnesium Sulfate (MgSO4)(MgSO_4)(MgSO4ΓÇï), Sodium Sulfate (Na2SO4)(Na_2SO_4)(Na2ΓÇïSO4ΓÇï), Zinc Sulfate (ZnSO4)(ZnSO_4)(ZnSO4ΓÇï)
144. Sodium Chloride (NaCl), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï)
145. Sodium Chloride (NaCl), Calcium Chloride (CaCl2)(CaCl_2)(CaCl2ΓÇï), Lithium Chloride (LiCl), Zinc Sulfate (ZnSO4)(ZnSO_4)(ZnSO4ΓÇï)
146. Sodium Chloride (NaCl), Calcium Chloride (CaCl2)(CaCl_2)(CaCl2ΓÇï), Sodium Sulfate (Na2SO4)(Na_2SO_4)(Na2ΓÇïSO4ΓÇï), Zinc Sulfate (ZnSO4)(ZnSO_4)(ZnSO4ΓÇï)
147. Sodium Chloride (NaCl), Lithium Chloride (LiCl), Sodium Sulfate (Na2SO4)(Na_2SO_4) (Na2ΓÇïSO4ΓÇï), Zinc Sulfate (ZnSO4)(ZnSO_4)(ZnSO4ΓÇï)
148. Potassium Chloride (KCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl)
149. Potassium Chloride (KCl), Magnesium Sulfate (MgSO4)(MgSO_4)(MgSO4ΓÇï), Calcium Chloride (CaCl2)(CaCl_2)(CaCl2ΓÇï), Sodium Sulfate (Na2SO4)(Na_2SO_4)(Na2ΓÇïSO4ΓÇï)
150. Potassium Chloride (KCl), Magnesium Sulfate (MgSO4)(MgSO_4)(MgSO4ΓÇï), Calcium Chloride (CaCl2)(CaCl_2)(CaCl2ΓÇï), Zinc Sulfate (ZnSO4)(ZnSO_4)(ZnSO4ΓÇï)
151. Potassium Chloride (KCl), Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï)
152. Potassium Chloride (KCl), Magnesium Sulfate (MgSO4)(MgSO_4)(MgSO4ΓÇï), Lithium Chloride (LiCl), Zinc Sulfate (ZnSO4)(ZnSO_4)(ZnSO4ΓÇï)
153. Potassium Chloride (KCl), Magnesium Sulfate (MgSO4)(MgSO_4)(MgSO4ΓÇï), Sodium Sulfate (Na2SO4)(Na_2SO_4)(Na2ΓÇïSO4ΓÇï), Zinc Sulfate (ZnSO4)(ZnSO_4)(ZnSO4ΓÇï)
154. Potassium Chloride (KCl), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï)
155. Potassium Chloride (KCl), Calcium Chloride (CaCl2)(CaCl_2)(CaCl2ΓÇï), Lithium Chloride (LiCl), Zinc Sulfate (ZnSO4)(ZnSO_4)(ZnSO4ΓÇï)
156. Potassium Chloride (KCl), Calcium Chloride (CaCl2)(CaCl_2)(CaCl2ΓÇï), Sodium Sulfate (Na2SO4)(Na_2SO_4)(Na2ΓÇïSO4ΓÇï), Zinc Sulfate (ZnSO4)(ZnSO_4)(ZnSO4ΓÇï)
157. Potassium Chloride (KCl), Lithium Chloride (LiCl), Sodium Sulfate (Na2SO4)(Na_2SO_4) (Na2ΓÇïSO4ΓÇï), Zinc Sulfate (ZnSO4)(ZnSO_4)(ZnSO4ΓÇï)
158. Magnesium Sulfate (MgSO4)(MgSO_4)(MgSO4ΓÇï), Calcium Chloride (CaCl2)(CaCl_2) (CaCl2ΓÇï), Lithium Chloride (LiCl), Sodium Sulfate (Na2SO4)(Na_2SO_4)(Na2ΓÇïSO4ΓÇï)
159. Magnesium Sulfate (MgSO4)(MgSO_4)(MgSO4ΓÇï), Calcium Chloride (CaCl2)(CaCl_2) (CaCl2ΓÇï), Lithium Chloride (LiCl), Zinc Sulfate (ZnSO4)(ZnSO_4)(ZnSO4ΓÇï)
160. Magnesium Sulfate (MgSO4)(MgSO_4)(MgSO4ΓÇï), Calcium Chloride (CaCl2)(CaCl_2) (CaCl2ΓÇï), Sodium Sulfate (Na2SO4)(Na_2SO_4)(Na2ΓÇïSO4ΓÇï), Zinc Sulfate (ZnSO4) (ZnSO_4)(ZnSO4ΓÇï)
161. Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)
162. Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)

Whereas 4 salts are combined and mixed with pharmaceutical grade solvent to create a saline solution reagent and added to a saliva sample containing one of the following proteins of interest which had previously been identified in the saliva sample by ICP-MS or ELISA assay.
Cytokeratin 19 fragment (CYFRA 21-1)
Tissue polypeptide antigen (TPS)
Cancer antigen 125 (CA-125)
CD44
Cathepsin V
Kallikrein 5
ADAM9
VEGF
EGF
CEA

The combination of saline solution plus saliva sample forces a unique crystal pattern which can be imaged, processed, identified and assigned a predetermined health condition which has been taught to the image processing artificial intelligence of the present invention.

### Five-Salt Combinations

163. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Calcium Chloride (CaCl2)(CaCl_2)(CaCl2ΓÇï)
164. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Lithium Chloride (LiCl)
165. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Sodium Sulfate (Na2SO4)(Na_2SO_4)(Na2ΓÇïSO4ΓÇï)
166. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Zinc Sulfate (ZnSO4)(ZnSO_4)(ZnSO4ΓÇï)
167. Ammonium Sulfate (NH4)2SO4(NH_4)_2SO_4(NH4ΓÇï)2ΓÇïSO4ΓÇï, Sodium Chloride (NaCl), Potassium Chloride (KCl), Calcium Chloride (CaCl2)(CaCl_2)(CaCl2ΓÇï), Lithium Chloride (LiCl)
168. Ammonium Sulfate (NH4)2SO4(NH_4)_2SO_4(NH4ΓÇï)2ΓÇïSO4ΓÇï, Sodium Chloride (NaCl), Potassium Chloride (KCl), Calcium Chloride (CaCl2)(CaCl_2)(CaCl2ΓÇï), Sodium Sulfate (Na2SO4)(Na_2SO_4)(Na2ΓÇïSO4ΓÇï)
169. Ammonium Sulfate (NH4)2SO4(NH_4)_2SO_4(NH4ΓÇï)2ΓÇïSO4ΓÇï, Sodium Chloride (NaCl), Potassium Chloride (KCl), Calcium Chloride (CaCl2)(CaCl_2)(CaCl2ΓÇï), Zinc Sulfate (ZnSO4)(ZnSO_4)(ZnSO4ΓÇï)
170. Ammonium Sulfate (NH4)2SO4(NH_4)_2SO_4(NH4ΓÇï)2ΓÇïSO4ΓÇï, Sodium Chloride (NaCl), Potassium Chloride (KCl), Lithium Chloride (LiCl), Sodium Sulfate (Na2SO4) (Na_2SO_4)(Na2ΓÇïSO4ΓÇï)
171. Ammonium Sulfate (NH4)2SO4(NH_4)_2SO_4(NH4γÇï)2γÇïSO4γÇï, Sodium Chloride (NaCl), Potassium Chloride (KCl), Lithium Chloride (LiCl), Zinc Sulfate (ZnSO4)(ZnSO_4) (ZnSO4ΓÇï)
172. Ammonium Sulfate (NH4)2SO4(NH_4)_2SO_4(NH4ΓÇï)2ΓÇïSO4ΓÇï, Sodium Chloride (NaCl), Potassium Chloride (KCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)
173. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Calcium Chloride (CaCl2) (CaCl_2)(CaCl2ΓÇï), Lithium Chloride (LiCl)
174. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Calcium Chloride (CaCl2) (CaCl_2)(CaCl2ΓÇï), Sodium Sulfate (Na2SO4)(Na_2SO_4)(Na2ΓÇïSO4ΓÇï)
175. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Calcium Chloride (CaCl2) (CaCl_2)(CaCl2ΓÇï), Zinc Sulfate (ZnSO4)(ZnSO_4)(ZnSO4ΓÇï)
176. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï)
177. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Lithium Chloride (LiCl), Zinc Sulfate (ZnSO4)(ZnSO_4)(ZnSO4ΓÇï)
178. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Sodium Sulfate (Na2SO4) (Na_2SO_4)(Na2ΓÇïSO4ΓÇï), Zinc Sulfate (ZnSO4)(ZnSO_4)(ZnSO4ΓÇï)
179. Ammonium Sulfate (NH4)2SO4(NH_4)_2SO_4(NH4ΓÇï)2ΓÇïSO4ΓÇï, Sodium Chloride (NaCl), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï)
180. Ammonium Sulfate (NH4)2SO4(NH_4)_2SO_4(NH4ΓÇï)2ΓÇïSO4ΓÇï, Sodium Chloride (NaCl), Calcium Chloride (CaCl2)(CaCl_2)(CaCl2ΓÇï), Lithium Chloride (LiCl), Zinc Sulfate (ZnSO4)(ZnSO_4)(ZnSO4ΓÇï)
181. Ammonium Sulfate (NH4)2SO4(NH_4)_2SO_4(NH4ΓÇï)2ΓÇïSO4ΓÇï, Sodium Chloride (NaCl), Calcium Chloride (CaCl2)(CaCl_2)(CaCl2ΓÇï), Sodium Sulfate (Na2SO4)(Na_2SO_4) (Na2ΓÇïSO4ΓÇï), Zinc Sulfate (ZnSO4)(ZnSO_4)(ZnSO4ΓÇï)
182. Ammonium Sulfate (NH4)2SO4(NH_4)_2SO_4(NH4ΓÇï)2ΓÇïSO4ΓÇï, Sodium Chloride (NaCl), Lithium Chloride (LiCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)
183. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl)
184. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Calcium Chloride (CaCl2)(CaCl_2)(CaCl2ΓÇï), Sodium Sulfate (Na2SO4)(Na_2SO_4)(Na2ΓÇïSO4ΓÇï)
185. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Calcium Chloride (CaCl2)(CaCl_2)(CaCl2ΓÇï), Zinc Sulfate (ZnSO4)(ZnSO_4)(ZnSO4ΓÇï)
186. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï)
187. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Lithium Chloride (LiCl), Zinc Sulfate (ZnSO4)(ZnSO_4)(ZnSO4ΓÇï)
188. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Sodium Sulfate (Na2SO4)(Na_2SO_4)(Na2ΓÇïSO4ΓÇï), Zinc Sulfate (ZnSO4)(ZnSO_4)(ZnSO4ΓÇï)
189. Ammonium Sulfate (NH4)2SO4(NH_4)_2SO_4(NH4ΓÇï)2ΓÇïSO4ΓÇï, Potassium Chloride (KCl), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï)
190. Ammonium Sulfate (NH4)2SO4(NH_4)_2SO_4(NH4ΓÇï)2ΓÇïSO4ΓÇï, Potassium Chloride (KCl), Calcium Chloride (CaCl2)(CaCl_2)(CaCl2ΓÇï), Lithium Chloride (LiCl), Zinc Sulfate (ZnSO4)(ZnSO_4)(ZnSO4ΓÇï)
191. Ammonium Sulfate (NH4)2SO4(NH_4)_2SO_4(NH4ΓÇï)2ΓÇïSO4ΓÇï, Potassium Chloride (KCl), Calcium Chloride (CaCl2)(CaCl_2)(CaCl2ΓÇï), Sodium Sulfate (Na2SO4) (Na_2SO_4)(Na2ΓÇïSO4ΓÇï), Zinc Sulfate (ZnSO4)(ZnSO_4)(ZnSO4ΓÇï)
192. Ammonium Sulfate (NH4)2SO4(NH_4)_2SO_4(NH4ΓÇï)2ΓÇïSO4ΓÇï, Potassium Chloride (KCl), Lithium Chloride (LiCl), Sodium Sulfate (Na2SO4)(Na_2SO_4) (Na2ΓÇïSO4ΓÇï), Zinc Sulfate (ZnSO4)(ZnSO_4)(ZnSO4ΓÇï)
193. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï)
194. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Calcium Chloride (CaCl2)(CaCl_2)(CaCl2ΓÇï), Lithium Chloride (LiCl), Zinc Sulfate (ZnSO4)(ZnSO_4)(ZnSO4ΓÇï)
195. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Calcium Chloride (CaCl2)(CaCl_2)(CaCl2ΓÇï), Sodium Sulfate (Na2SO4)(Na_2SO_4)(Na2ΓÇïSO4ΓÇï), Zinc Sulfate (ZnSO4)(ZnSO_4) (ZnSO4ΓÇï)
196. Ammonium Sulfate (NH4)2SO4(NH_4)_2SO_4(NH4ΓÇï)2ΓÇïSO4ΓÇï, Magnesium Sulfate (MgSO4)(MgSO_4)(MgSO4ΓÇï), Lithium Chloride (LiCl), Sodium Sulfate (Na2SO4) (Na_2SO_4)(Na2ΓÇïSO4ΓÇï), Zinc Sulfate (ZnSO4)(ZnSO_4)(ZnSO4ΓÇï)
197. Ammonium Sulfate (NH4)2SO4(NH_4)_2SO_4(NH4ΓÇï)2ΓÇïSO4ΓÇï, Calcium Chloride (CaCl2)(CaCl_2)(CaCl2ΓÇï), Lithium Chloride (LiCl), Sodium Sulfate (Na2SO4)(Na_2SO_4) (Na2ΓÇïSO4ΓÇï), Zinc Sulfate (ZnSO4)(ZnSO_4)(ZnSO4ΓÇï)
198. Sodium Chloride (NaCl), Potassium Chloride (KCl), Magnesium Sulfate (MgSO4) (MgSO_4)(MgSO4ΓÇï), Calcium Chloride (CaCl2)(CaCl_2)(CaCl2ΓÇï), Lithium Chloride (LiCl)
199. Sodium Chloride (NaCl), Potassium Chloride (KCl), Magnesium Sulfate (MgSO4) (MgSO_4)(MgSO4ΓÇï), Calcium Chloride (CaCl2)(CaCl_2)(CaCl2ΓÇï), Sodium Sulfate (Na2SO4)(Na_2SO_4)(Na2ΓÇïSO4ΓÇï)
200. Sodium Chloride (NaCl), Potassium Chloride (KCl), Magnesium Sulfate (MgSO4) (MgSO_4)(MgSO4ΓÇï), Calcium Chloride (CaCl2)(CaCl_2)(CaCl2ΓÇï), Zinc Sulfate (ZnSO4) (ZnSO_4)(ZnSO4ΓÇï)
201. Sodium Chloride (NaCl), Potassium Chloride (KCl), Magnesium Sulfate (MgSO4) (MgSO_4)(MgSO4ΓÇï), Lithium Chloride (LiCl), Sodium Sulfate (Na2SO4)(Na_2SO_4) (Na2ΓÇïSO4ΓÇï)
202. Sodium Chloride (NaCl), Potassium Chloride (KCl), Magnesium Sulfate (MgSO4) (MgSO_4)(MgSO4ΓÇï), Lithium Chloride (LiCl), Zinc Sulfate (ZnSO4)(ZnSO_4)(ZnSO4ΓÇï)
203. Sodium Chloride (NaCl), Potassium Chloride (KCl), Magnesium Sulfate (MgSO4) (MgSO_4)(MgSO4ΓÇï), Sodium Sulfate (Na2SO4)(Na_2SO_4)(Na2ΓÇïSO4ΓÇï), Zinc Sulfate (ZnSO4)(ZnSO_4)(ZnSO4ΓÇï)
204. Sodium Chloride (NaCl), Potassium Chloride (KCl), Calcium Chloride (CaCl2)(CaCl_2) (CaCl2ΓÇï), Lithium Chloride (LiCl), Sodium Sulfate (Na2SO4)(Na_2SO_4)(Na2ΓÇïSO4ΓÇï)
205. Sodium Chloride (NaCl), Potassium Chloride (KCl), Calcium Chloride (CaCl2)(CaCl_2) (CaCl2ΓÇï), Lithium Chloride (LiCl), Zinc Sulfate (ZnSO4)(ZnSO_4)(ZnSO4ΓÇï)
206. Sodium Chloride (NaCl), Potassium Chloride (KCl), Calcium Chloride (CaCl2)(CaCl_2) (CaCl2ΓÇï), Sodium Sulfate (Na2SO4)(Na_2SO_4)(Na2ΓÇïSO4ΓÇï), Zinc Sulfate (ZnSO4) (ZnSO_4)(ZnSO4ΓÇï)
207. Sodium Chloride (NaCl), Potassium Chloride (KCl), Lithium Chloride (LiCl), Sodium Sulfate (Na2SO4)(Na_2SO_4)(Na2ΓÇïSO4ΓÇï), Zinc Sulfate (ZnSO4)(ZnSO_4)(ZnSO4ΓÇï) 208. Sodium Chloride (NaCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl), Sodium Sulfate (Na2SO4) (Na_2SO_4)(Na2ΓÇïSO4ΓÇï)
209. Sodium Chloride (NaCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl), Zinc Sulfate (ZnSO4)(ZnSO_4) (ZnSO4ΓÇï)
210. Sodium Chloride (NaCl), Magnesium Sulfate (MgSO4)(MgSO_4)(MgSO4ΓÇï), Calcium Chloride (CaCl2)(CaCl_2)(CaCl2ΓÇï), Sodium Sulfate (Na2SO4)(Na_2SO_4) (Na2ΓÇïSO4ΓÇï), Zinc Sulfate (ZnSO4)(ZnSO_4)(ZnSO4ΓÇï)
211. Sodium Chloride (NaCl), Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï), Zinc Sulfate (ZnSO4) (ZnSO_4)(ZnSO4ΓÇï)
212. Sodium Chloride (NaCl), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï), Zinc Sulfate (ZnSO4) (ZnSO_4)(ZnSO4ΓÇï)
213. Potassium Chloride (KCl), Magnesium Sulfate (MgSO4)(MgSO_4)(MgSO4γÇï), Calcium Chloride (CaCl2)(CaCl_2)(CaCl2ΓÇï), Lithium Chloride (LiCl), Sodium Sulfate (Na2SO4) (Na_2SO_4)(Na2ΓÇïSO4ΓÇï)
214. Potassium Chloride (KCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl), Zinc Sulfate (ZnSO4)(ZnSO_4) (ZnSO4ΓÇï)
215. Potassium Chloride (KCl), Magnesium Sulfate (MgSO4)(MgSO_4)(MgSO4ΓÇï), Calcium Chloride (CaCl2)(CaCl_2)(CaCl2ΓÇï), Sodium Sulfate (Na2SO4)(Na_2SO_4) (Na2ΓÇïSO4ΓÇï), Zinc Sulfate (ZnSO4)(ZnSO_4)(ZnSO4ΓÇï)
216. Potassium Chloride (KCl), Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï), Zinc Sulfate (ZnSO4) (ZnSO_4)(ZnSO4ΓÇï)
217. Potassium Chloride (KCl), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï), Zinc Sulfate (ZnSO4) (ZnSO_4)(ZnSO4ΓÇï)
218. Magnesium Sulfate (MgSO4)(MgSO_4)(MgSO4ΓÇï), Calcium Chloride (CaCl2)(CaCl_2) (CaCl2ΓÇï), Lithium Chloride (LiCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)

Whereas 5 salts are combined and mixed with pharmaceutical grade solvent to create a saline solution reagent and added to a saliva sample containing one of the following proteins of interest which had previously been identified in the saliva sample by ICP-MS or ELISA assay.
Cytokeratin 19 fragment (CYFRA 21-1)
Tissue polypeptide antigen (TPS)
Cancer antigen 125 (CA-125)
CD44
Cathepsin V
Kallikrein 5
ADAM9
VEGF
EGF
CEA

The combination of saline solution plus saliva sample forces a unique crystal pattern which can be imaged, processed, identified and assigned a predetermined health condition which has been taught to the image processing artificial intelligence of the present invention.

### Six-Salt Combinations

219. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl)
220. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Calcium Chloride (CaCl2)(CaCl_2)(CaCl2ΓÇï), Sodium Sulfate (Na2SO4)(Na_2SO_4) (Na2ΓÇïSO4ΓÇï)
221. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Calcium Chloride (CaCl2)(CaCl_2)(CaCl2ΓÇï), Zinc Sulfate (ZnSO4)(ZnSO_4)(ZnSO4ΓÇï)
222. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï)
223. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Lithium Chloride (LiCl), Zinc Sulfate (ZnSO4)(ZnSO_4)(ZnSO4ΓÇï)
224. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Sodium Sulfate (Na2SO4)(Na_2SO_4)(Na2ΓÇïSO4ΓÇï), Zinc Sulfate (ZnSO4)(ZnSO_4) (ZnSO4ΓÇï)
225. Ammonium Sulfate (NH4)2SO4(NH_4)_2SO_4(NH4ΓÇï)2ΓÇïSO4ΓÇï, Sodium Chloride (NaCl), Potassium Chloride (KCl), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï)
226. Ammonium Sulfate (NH4)2SO4(NH_4)_2SO_4(NH4ΓÇï)2ΓÇïSO4ΓÇï, Sodium Chloride (NaCl), Potassium Chloride (KCl), Calcium Chloride (CaCl2)(CaCl_2)(CaCl2ΓÇï), Lithium Chloride (LiCl), Zinc Sulfate (ZnSO4)(ZnSO_4)(ZnSO4ΓÇï)
227. Ammonium Sulfate (NH4)2SO4(NH_4)_2SO_4(NH4ΓÇï)2ΓÇïSO4ΓÇï, Sodium Chloride (NaCl), Potassium Chloride (KCl), Calcium Chloride (CaCl2)(CaCl_2)(CaCl2ΓÇï), Sodium Sulfate [symbol] á(Na2SO4)(Na_2SO_4)(Na2ΓÇïSO4ΓÇï), Zinc Sulfate (ZnSO4)(ZnSO_4)(ZnSO4ΓÇï)
228. Ammonium Sulfate (NH4)2SO4(NH_4)_2SO_4(NH4ΓÇï)2ΓÇïSO4ΓÇï, Sodium Chloride (NaCl), Potassium Chloride (KCl), Lithium Chloride (LiCl), Sodium Sulfate (Na2SO4) (Na_2SO_4)(Na2ΓÇïSO4ΓÇï), Zinc Sulfate (ZnSO4)(ZnSO_4)(ZnSO4ΓÇï)
229. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Calcium Chloride (CaCl2) (CaCl_2)(CaCl2ΓÇï), Lithium Chloride (LiCl), Sodium Sulfate (Na2SO4)(Na_2SO_4) (Na2ΓÇïSO4ΓÇï)
230. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Calcium Chloride (CaCl2) (CaCl_2)(CaCl2ΓÇï), Lithium Chloride (LiCl), Zinc Sulfate (ZnSO4)(ZnSO_4)(ZnSO4ΓÇï)
231. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Calcium Chloride (CaCl2) (CaCl_2)(CaCl2ΓÇï), Sodium Sulfate (Na2SO4)(Na_2SO_4)(Na2ΓÇïSO4ΓÇï), Zinc Sulfate (ZnSO4)(ZnSO_4)(ZnSO4ΓÇï)
232. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)
233. Ammonium Sulfate (NH4)2SO4(NH_4)_2SO_4(NH4ΓÇï)2ΓÇïSO4ΓÇï, Sodium Chloride (NaCl), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)
234. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl), Sodium Sulfate (Na2SO4)(Na_2SO_4) (Na2ΓÇïSO4ΓÇï)
235. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl), Zinc Sulfate (ZnSO4)(ZnSO_4) (ZnSO4ΓÇï)
236. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Calcium Chloride (CaCl2)(CaCl_2)(CaCl2ΓÇï), Sodium Sulfate (Na2SO4)(Na_2SO_4)(Na2ΓÇïSO4ΓÇï), Zinc Sulfate (ZnSO4)(ZnSO_4)(ZnSO4ΓÇï)
237. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï), Zinc Sulfate (ZnSO4)(ZnSO_4) (ZnSO4ΓÇï)
238. Ammonium Sulfate (NH4)2SO4(NH_4)_2SO_4(NH4ΓÇï)2ΓÇïSO4ΓÇï, Potassium Chloride (KCl), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï), Zinc Sulfate (ZnSO4)(ZnSO_4) (ZnSO4ΓÇï)

Whereas 6 salts are combined and mixed with pharmaceutical grade solvent to create a saline solution reagent and added to a saliva sample containing one of the following proteins of interest which had previously been identified in the saliva sample by ICP-MS or ELISA assay.
Cytokeratin 19 fragment (CYFRA21-1)
Tissue polypeptide antigen (TPS)
Cancer antigen 125 (CA-125 )
CD44
Cathepsin V
Kallikrein 5
ADAM9
VEGF
EGF
CEA

The combination of saline solution plus saliva sample forces a unique crystal pattern which can be imaged, processed, identified and assigned a predetermined health condition which has been taught to the image processing artificial intelligence of the present invention.

### Seven-Salt Combinations

239. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï)
240. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl), Zinc Sulfate (ZnSO4) (ZnSO_4)(ZnSO4ΓÇï)
241. Ammonium Sulfate (NH4)2SO4(NH_4)_2SO_4(NH4γÇï)2γÇïSO4γÇï, Sodium Chloride (NaCl), Potassium Chloride (KCl), Magnesium Sulfate (MgSO4)(MgSO_4)(MgSO4ΓÇï), Calcium Chloride (CaCl2)(CaCl_2)(CaCl2ΓÇï), Sodium Sulfate (Na2SO4)(Na_2SO_4) (Na2ΓÇïSO4ΓÇï), Zinc Sulfate (ZnSO4)(ZnSO_4)(ZnSO4ΓÇï)
242. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Lithium Chloride (LiCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)
243. Ammonium Sulfate (NH4)2SO4(NH_4)_2SO_4(NH4ΓÇï)2ΓÇïSO4ΓÇï, Sodium Chloride (NaCl), Potassium Chloride (KCl), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï), Zinc Sulfate (ZnSO4) (ZnSO_4)(ZnSO4ΓÇï)
244. Ammonium Sulfate (NH4)2SO4(NH_4)_2SO_4(NH4ΓÇï)2ΓÇïSO4ΓÇï, Sodium Chloride (NaCl), Magnesium Sulfate (MgSO4)(MgSO_4)(MgSO4ΓÇï), Calcium Chloride (CaCl2) (CaCl_2)(CaCl2ΓÇï), Lithium Chloride (LiCl), Sodium Sulfate (Na2SO4)(Na_2SO_4) (Na2ΓÇïSO4ΓÇï), Zinc Sulfate (ZnSO4)(ZnSO_4)(ZnSO4ΓÇï)
245. Ammonium Sulfate (NH4)2SO4(NH_4)_2SO_4(NH4ΓÇï)2ΓÇïSO4ΓÇï, Potassium Chloride (KCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl), Sodium Sulfate (Na2SO4)(Na_2SO_4) (Na2ΓÇïSO4ΓÇï), Zinc Sulfate (ZnSO4)(ZnSO_4)(ZnSO4ΓÇï)

Whereas 7 salts are combined and mixed with pharmaceutical grade solvent to create a saline solution reagent and added to a saliva sample containing one of the following proteins of interest which had previously been identified in the saliva sample by ICP-MS or ELISA assay.
Cytokeratin 19 fragment (CYFRA 21-1)
Tissue polypeptide antigen (TPS)
Cancer antigen 125 (CA-125)
CD44
Cathepsin V
Kallikrein 5
ADAM9
VEGF
EGF
CEA

The combination of saline solution plus saliva sample forces a unique crystal pattern which can be imaged, processed, identified and assigned a predetermined health condition which has been taught to the image processing artificial intelligence of the present invention.

### Eight-Salt Combination

246. Ammonium Sulfate (NH4)2SO4(NH_4)_2SO_4(NH4ΓÇï)2ΓÇïSO4ΓÇï, Sodium Chloride (NaCl), Potassium Chloride (KCl), Magnesium Sulfate (MgSO4)(MgSO_4)(MgSO4ΓÇï), Calcium Chloride (CaCl2)(CaCl_2)(CaCl2ΓÇï), Lithium Chloride (LiCl), Sodium Sulfate (Na2SO4)(Na_2SO_4)(Na2ΓÇïSO4ΓÇï), Zinc Sulfate (ZnSO4)(ZnSO_4)(ZnSO4ΓÇï)

Whereas 8 salts are combined and mixed with a pharmaceutical grade solvent to create a saline solution reagent and added to a saliva sample containing one of the following proteins of interest which had previously been identified in the saliva sample by ICP-MS or ELISA assay.
Cytokeratin 19 fragment (CYFRA 21-1)
Tissue polypeptide antigen (TPS)
Cancer antigen 125 (CA-125 )
CD44
Cathepsin V
Kallikrein 5
ADAM9
VEGF
EGF
CEA

The combination of saline solution plus saliva sample forces a unique crystal pattern which can be imaged, processed, identified and assigned a predetermined health condition which has been taught to the image processing artificial intelligence of the present invention.

In protein precipitation processes, particularly during “salting out,” pH modifiers play a critical role in optimizing protein solubility and stability, enhancing the precipitation process. Hydrochloric acid (HCl) is commonly used to lower the pH by protonating acidic groups on proteins, thereby reducing their solubility and promoting precipitation. Similarly, sodium hydroxide (NaOH) is employed to increase pH, deprotonating acidic groups and neutralizing excess acid to create optimal ionic conditions. Acetic acid, a mild acid, is often preferred for gently lowering pH, while ammonium hydroxide (NH₄OH) is useful for raising pH in conjunction with ammonium sulfate, particularly in alkaline conditions. Sodium acetate, acting as both a buffering agent and a pH modifier, maintains stable pH levels during salting out and is frequently paired with ammonium sulfate. Phosphoric acid (H₃PO₄) is another option for lowering pH and is commonly used in acidic buffer systems for protein precipitation. Citric acid, also a mild acid, provides a similar function and is often included in citrate buffer systems to stabilize proteins. For increasing pH, potassium hydroxide (KOH) is sometimes used as an alternative to NaOH, contributing potassium ions that may further enhance precipitation. Buffers like Tris (tris(hydroxymethyl)aminomethane) are widely utilized for maintaining stable pH in neutral to slightly alkaline ranges, while MES (2-(N-Morpholino) ethanesulfonic acid) buffers are ideal for mildly acidic conditions. HEPES (4-(2-Hydroxyethyl)piperazine-1-ethanesulfonic acid) provides stable pH in neutral to slightly alkaline conditions and is particularly suited for biological systems. Lastly, boric acid (H₃BO₃) is used to adjust pH in borate buffer systems, effective for proteins requiring pH ranges between 7.5 and 9.0. Each of these pH modifiers is strategically selected based on the specific requirements of the target protein, with adjustments often guided by the protein's isoelectric point (pIpIpI), ensuring the most efficient precipitation environment.

Therefore, in certain embodiments of the present invention one PH modifier or PH buffer solution, or a plurality of PH modifiers are used in the process of salting out specific proteins of interest in addition to saline solutions as described by prior embodiments of the present invention. PH modifiers have been observed to either enhance protein crystal patterns or diminish protein crystal patterns, depending on specific formulations of saline solution, combinations of salts in saline solutions, solvents used in the manufacturing of saline solutions, PH modifiers and PH buffers, and proteins involved in the precipitation process. Diminishing protein crystal patterns in dried saliva is a useful tool for reduction of background noise when attempting to visualize a particular protein crystal pattern of interest. Similarly, enhancing protein crystal patterns allows for clearer imaging and processing by the digital platform of the present invention.

In salting-out processes, the choice of solvent is critical for dissolving salts, creating saline solutions, and promoting effective protein precipitation. Water (H₂O) is the most commonly used solvent due to its high polarity and ability to dissolve a wide variety of salts, providing a stable medium for ionic interactions. Ethanol (CH₃CH₂OH) is often added as a co-solvent to reduce water activity, destabilize hydration shells, and enhance protein precipitation. Similarly, methanol (CH₃OH) and isopropanol (C₃H₈O) are used to disrupt water-protein interactions, with isopropanol being particularly effective in co-precipitation processes for proteins and DNA. Acetone (C₃H₆O) is another option, commonly used to rapidly precipitate proteins by reducing water's dielectric constant. Buffer solutions, such as phosphate or Tris buffers, are frequently employed to maintain a stable pH environment and ensure protein stability during the salting-out process. Ammonium sulfate solutions are unique in serving as both a solvent and a salting-out agent due to their high solubility, enabling selective and gradual protein precipitation. Dimethyl sulfoxide (DMSO) is occasionally utilized for hydrophobic proteins or those with limited solubility in water, while glycerol (C3H8O3) can be included to preserve protein structure and reduce denaturation. The selection of solvents and co-solvents depends on factors such as polarity, protein stability, and compatibility with downstream processes, with water often forming the foundation of saline solutions enhanced by additives like ethanol or ammonium sulfate for optimal results.

Therefore, in certain embodiments of the present invention a plurality of solvents plus salts are employed in the production of saline solution. Similarly, other embodiments require a single solvent plus salts combination. Finally, certain embodiments of the present invention require a combination of solvents plus salts plus PH modifiers or PH buffer solutions as described herein.

### REFERENCES

SpringerLink. [symbol] áPhysiological regulation of oral saliva ion composition and flow rate. [symbol] áLink
Cleveland Clinic. [symbol] áRenin-Angiotensin-Aldosterone System (RAAS). [symbol] áLink
CEGH. [symbol] áDoes the salivary fern pattern determine fertile period in reproductive women? [symbol] áLink
Verywell Health. [symbol] áSalivary Glands: Anatomy, Function, and Conditions. [symbol] áLink
Jaypee Digital. [symbol] áEvaluation of Salivary Ferning for Predicting Ovulation in Menstruating Women. [symbol] áLink
Cleveland Clinic—Renin-Angiotensin-Aldosterone System (RAAS) https://my.clevelandclinic.org/health/articles/24175-renin-angiotensin-aldosterone-system-raas
Cleveland Clinic—Cystic Fibrosis https://my.clevelandclinic.org/health/diseases/15315-cystic-fibrosis
Cleveland Clinic—Hyperaldosteronism https://my.clevelandclinic.org/health/diseases/16448-hyperaldosteronism
Johns Hopkins Sj [symbol] grenΓÇÖs Center-Sj [symbol] grenΓÇÖs Disease Information https://www.hopkinssjogrens.org/disease-information/
Medicinenet—How to Tell if You Are Dehydrated https://www.medicinenet.com/how_can_you_tell_if_you_are_dehydrated/article. htm
National Kidney Foundation—About Chronic Kidney Disease https://www.kidney.org/atoz/content/about-chronic-kidney-disease
Cleveland Clinic—PCOS (Polycystic Ovary Syndrome) https://my.clevelandclinic.org/health/diseases/12126-polycystic-ovary-syndrome-pcos
TeachMePhysiology—The Renin-Angiotensin-Aldosterone System https://teachmephysiology.com/urinary-system/regulation/the-renin-angiotensin-aldosterone-system/
UpToDate—Bacterial Vaginosis: The Basics https://www.uptodate.com/contents/bacterial-vaginosis-the-basics
Jaypee Digital—Evaluation of Salivary Ferning for Predicting Ovulation in Menstruating Women https://www.jaypeedigital.com/abstractArticleContentBrowse/JSAFOG/33252/JPJ/fullText
Cleveland Clinic—AddisonΓÇÖs Disease https://my.clevelandclinic.org/health/diseases/10079-addisons-disease
Johns Hopkins Medicine—Salivary Gland Cancer https://www.hopkinsmedicine.org/health/conditions-and-diseases/salivary-gland-cancer

Enhancing biomarker detection in human serum for lung cancer diagnosis: Aqueous biphasic systems for simultaneous depletion of high-abundance proteins and efficient extraction of CYFRA 21ΓÇô1 https://www.sciencedirect.com/science/article/pii/S 2772582024000159

What is claimed is:

1. A method of establishing diagnostic protocols utilizing a ferning image of a bodily fluid secretion, comprising:
obtaining a sample of the bodily fluid secretion;
cataloging ferning images for known health conditions and bodily fluid types to create an accessible database of ferning images correlated with known health issues;
forming a sample ferning pattern from said sample of the bodily fluid secretion; and
comparing said sample ferning pattern with said ferning images in said accessible database.

2. A method according to claim 1, wherein said comparing includes applying AI to accurately discern nuances of the ferning patterns in said accessible database and that of the sample ferning pattern to determine a match.

3. A method according to claim 2, further comprising:
adding a selected saline based excipient from sets of saline choices to force a creation of a ferning pattern indicative of a normal physiological state or a presence of adverse health conditions.

4. A method according to claim 3, wherein said bodily fluid comprises at least one of saliva, mucous or perpetration.

5. A system for qualitative and quantitative analysis of a bodily fluid including therein excreted electrolytes, comprising:
an optical imaging analyzer capable of recording ferning crystallization patterns of dried bodily fluid samples, including stored collected images correlated to at least one of known physical states or adverse health conditions;
a catalog of ferning crystallization patterns correlated to known physical states or adverse health conditions previously diagnosed in human subjects; and
an artificial intelligence processor on which is stored an image recognition and processing algorithm capable of discerning otherwise imperceivable differences in crystal patterns found in dried bodily fluid ferning crystallization.

6. A process for determining presence and characteristics of at least one sample ferning pattern obtained from a sample of a secreted bodily fluid, comprising:
providing an optical analyzer operable to capture an image of said sample ferning pattern;
creating a database of ferning images correlated to stored parameters descriptive of at least one of bodily fluid type, adverse health issue or saline type;
forming a sample ferning pattern from said at least one sample ferning pattern of the bodily fluid secretion; and
comparing said sample ferning pattern with said ferning images in said accessible database.

7. A process according to claim 6, further comprising adding a selected saline based excipient from sets of various saline choices to force a creation of a ferning pattern indicative of a normal physiological state or a presence of a adverse health conditions.

8. A process according to claim 7, further comprising an AI processor capable of accurately discerning nuances of the ferning patterns in said database and that of the sample ferning pattern to determine a match.

9. The process according to claim 8, effective to visually demonstrate existence of proteins of interest through forced crystallization comprised of salt based excipient sets at least one cancer of the group of lung, oral and gastric cancers.

10. Products by the process of claim 9, comprised of unique crystalline structures susceptible of visually differentiated depictions.

11. The process according to claim 9, further comprised of: reagent sets, driving screening from the groups set forth within the instant specification.

12. The process according to claim 11, wherein mapping from the proteins of interest to disease states and diagnostics using the above process and salt complexes set forth via the combination of saline solution plus a saliva sample forces a unique crystal pattern which can be imaged, processed, identified and assigned a predetermined health condition based upon at least one of a single salt combinations, a double salt combinations, a triple salt combination, a four salt combinations, and a five or more salt combinations.

13. The process according to claim 11, the combination of saline solution plus a saliva sample forces a unique crystal pattern which can be imaged, processed, identified and assigned a predetermined health condition based upon at least one of the six salt combinations, as set forth herein and a seven salt combinations, as set forth herein.
